(19) United States
(12) Patent Application Publication
(10) Pub. No.: US 2024/0409868 A1
(43) Pub. Date: Dec. 12, 2024

(54) BIOREACTORS PROVIDING CERIUM OXIDE AND ZIRCONIUM DIOXIDE

(71) Applicant: XHEME, INC., Newton, MA (US)

(72) Inventor: Siva Sai Ramana Kumar CHALLA, Newton, MA (US)

(21) Appl. No.: 18/737,021

(22) Filed: Jun. 7, 2024

Related U.S. Application Data
(60) Provisional application No. 63/507,312, filed on Jun. 9, 2023.

(51) Int. Cl. C12M 1/12

(52) U.S. Cl. CPC C12M 25/16

(57) ABSTRACT

Cerium oxide and zirconium dioxide for biomanufacturing. More specifically, cerium oxide nanoparticles and/or nanoporous cerium oxide nanoparticle macro-structures and/or zirconium dioxide nanoparticles are provided in a bioreactor for biomanufacturing and/or to promote cellular growth/expansion.

### FIELD

The present disclosure is directed at cerium oxide and zirconium dioxide for biomanufacturing. More specifically, cerium oxide nanoparticles and/or nanoporous cerium oxide nanoparticle macro-structures and/or zirconium dioxide nanoparticles are provided in a bioreactor for biomanufacturing and/or to promote cellular growth/expansion.

### BACKGROUND

Biomanufacturing or bioprocessing is the production of biological products from living cells. A bioprocess is a specific process that uses complete living cells or their components (e.g., bacteria, enzymes, chloroplasts) to obtain desired products. The transport of energy and mass is fundamental to many biological and environmental processes. End products can be anything from biofuels produced from microalgae or antibiotics or pigments or chemicals produced or created from micromold, such as penicillin. Beer produced from yeast is another example of bioprocessing. Production of biological products from cells is reported to be negatively impacted by cellular stress. Such cellular stress may then lead to a decrease in cell viability and therefore, a reduction in yield and the quality of the biological products produced. See, e.g, Oxidative Stress-Alleviating Strategies To Improve Recombinant Protein Production In CHO Cells, Biotechnol Bioeng, 2020 April; 117 (4); 1172-1186.

Cells under environmental stress are reported to produce hydrogen peroxide or in general Reactive Oxygen Species (ROS). Accordingly, a need exists to provide mediums that more effectively produce oxygen from ROS or scavenge the free radicals, and in particular mediums that convert the hydrogen peroxide and/or ROS to oxygen, to mitigate oxidative stress on the cells employed for biomanufacturing and to increase cell viability. Similarly, a need exists to provide such mediums that can be employed for cellular growth/expansion.

### SUMMARY

A method of producing at least one biologically-produced product comprising providing a cell culture medium and at least a first type of biological cell configured to express at least one biologically-produced product, placing the cell culture medium and said first type of biological cell in a bioreactor wherein the bioreactor provides at least one of cerium oxide nanoparticles, nanoporous cerium oxide nanoparticle macro-structures, or zirconium dioxide nanoparticles, and causing the first type of biological cells to express the at least one biological product.

A method of producing at least one biologically-produced product comprising providing a cell culture medium and at least a first type of biological cell configured to express at least one biologically-produced product, placing the cell culture medium and the first type of biological cell in a bioreactor comprising polymer film containing cerium oxide nanoparticles and/or nanoporous cerium oxide nanoparticle macro-structures and/or zirconium dioxide nanoparticles, and causing the first type of biological cell to express the at least one biological product.

A method for growth/expansion of at least one biological cell comprising providing a cell culture medium and at least a first type of biological cell for growth/expansion, placing the cell culture medium and the first type of biological cell in a bioreactor wherein the bioreactor provides at least one of cerium oxide nanoparticles, nanoporous cerium oxide nanoparticle macro-structures, or zirconium dioxide nanoparticles, and causing the first type of biological cell to undergo growth/expansion.

A biomanufacturing system comprising a bioreactor providing at least one of cerium oxide nanoparticles, nanoporous cerium oxide nanoparticle macro-structures, or zirconium dioxide nanoparticles, a cell culture medium and at least a first type of biological cell contained in the bioreactor configured to express at least one biologically-produced product. The cerium oxide nanoparticles and/or nanoporous cerium oxide nanoparticle macro-structures and/or zirconium dioxide nanoparticles are configured to convert reactive oxygen species within the cell culture medium to oxygen and/or scavenge free radicals, such as those made during cell metabolism.

A biomanufacturing system comprising a bioreactor containing a cell culture medium and at least a first type of biological cell configured to express at least one biologically-produced product, the bioreactor including a polymer film containing at least one of cerium oxide nanoparticles, nanoporous cerium oxide nanoparticle macro-structures or zirconium dioxide nanoparticles. The polymer film containing cerium oxide nanoparticles and/or nanoporous cerium oxide nanoparticle macro-structures and/or zirconium dioxide nanoparticles is configured to contact said cell culture medium and convert reactive oxygen species within the cell culture medium to oxygen and/or scavenge free radicals, such as those made during cell metabolism. The polymer film may form the bioreactor to contain the cell culture medium.

A bioreactor comprising polymer film containing at least one of cerium oxide nanoparticles, nanoporous cerium oxide nanoparticle macro-structures or zirconium dioxide nanoparticles.

### BRIEF DESCRIPTION OF THE DRAWINGS

Aspects of the present disclosure may be appreciated upon review of the description herein and the accompanying drawings which identify as follows:

FIG. 1 illustrates cerium oxide nanoparticles;

FIG. 2 illustrates the nanoporous cerium oxide nanoparticle (NCeONP) macro-structure formed from the cerium oxide nanoparticles illustrated in FIG. 1;

FIG. 3 is a scanning electron micrograph of the cerium oxide nanoparticles employed to form the nanoporous cerium oxide nanoparticle macro-structure;

FIG. 4A is a scanning electron micrograph of the nanoporous cerium oxide nanoparticle macro-structure at the indicated magnification;

FIG. 4B is another scanning electron micrograph of the nanoporous cerium oxide nanoparticle macro-structure at the indicated magnification;

FIG. 4C is another scanning electron micrograph of the nanoporous cerium oxide nanoparticle macro-structure at the indicated magnification;

FIG. 5 provides the oxygen mapping of media both with and without plasticized poly(vinyl chloride) that contains 5.0 wt. % of nanoporous cerium oxide nanoparticle macro-structures;

FIG. 6 provides the oxygen mapping of cells in media both with and without the plasticized poly(vinyl chloride) film that contained 5.0 wt. % of nanoporous cerium oxide nanoparticle macro-structures;

FIG. 7A is a plot of proliferating cells (arbitrary values) of 10K HEK 293 in the presence of plasticized PVC films containing 5.0 wt. % nanoporous cerium oxide nanoparticle macro-structures at 1 hr, 24 hr and 48 hr. as compared to a control film (plasticized PVC film without any nanoporous cerium oxide nanoparticle macro-structures;

FIG. 7B is a plot of percentage viability at 24 hrs. for the control plasticized PVC film and the plasticized PVC film containing 5.0 wt. % nanoporous cerium oxide nanoparticle macro-structures;

FIG. 7C is a plot of the proliferating cells (arbitrary values) of 10K HEK 293 in the presence of polyethylene film containing 2.0 wt. % nanoporous cerium oxide nanoparticle macro-structures and zirconium dioxide nanoparticles at a 1:1 ratio, at 1 hr, 24 hr and 48 hr. as compared to a control film (polyethylene) without any nanoporous cerium oxide nanoparticle macro-structures and zirconium dioxide nanoparticles;

FIG. 7D is a plot of percentage viability at 24 hrs. for the control polyethylene film and the polyethylene film containing 2.0 wt. % nanoporous cerium oxide nanoparticle macro-structures and zirconium dioxide nanoparticles at a 1:1 ratio;

FIG. 8 is a SEM showing the morphology of the HEK cells after 48 hours in the presence of plasticized PVC films containing 5.0 wt. % nanoporous cerium oxide nanoparticle macro-structures versus a control (plasticized PVC film);

FIG. 9 is a SEM showing the morphology of the HEK cells after 48 hours in the presence of polyethylene film containing 2.0 wt. % nanoporous cerium oxide nanoparticle macro-structures and zirconium dioxide nanoparticles at a 1:1 ratio;

FIG. 10 is a plot of dissolved oxygen (DO) versus time for the identified films;

FIG. 11 is a plot of dissolved oxygen (DO) versus time showing the DO generation increase with the weight of the film;

FIG. 12A is a plot of dissolved oxygen (DO) versus time showing the DO generation increase with an increase in the weight of the film;

FIG. 12B is a plot of dissolved oxygen (DO) versus time shown DO generation by ethylene vinyl acetate (EVA) compounded with the various indicated additives;

FIG. 13A is a plot of dissolved oxygen (DO) versus time for the following when placed in 100 ml water solution containing 10 ml of 3.0% hydrogen peroxide: a particulate form (100 mg) of nanoporous cerium oxide nanoparticle (NCeONP) macrostructures; zirconium dioxide nanoparticles; a particulate mixture of nanoporous cerium oxide nanoparticle macro-structures and zirconium dioxide nanoparticles at a 1:1 ratio;

FIG. 13B shows the cell viability versus concentration for nanoporous cerium oxide nanoparticle (NCeONP) macrostructures in a 1:1 mixture with zirconium dioxide nanoparticles, nanoporous cerium oxide nanoparticle (NCeONP) macro-structures, and zirconium dioxide, when placed in a cell culture medium to evaluate the cell viability of HEK293F cells;

FIG. 14 is a plot of cell growth for a 1:1 mixture of NCeONP and ZrO₂ for a control (“0’) and at 617 μm and 1852 μm of the 1:1 mixture of NCeONP and ZrO₂. The growth of cells was measured on day 3 in a 96-well plate over the three days;

FIG. 15 is a plot of the Integral Variable Cell Density (IVCD) for “A”, a 1:1 mixture of NCeONP and ZrO₂ at 0 μm (control) and for “B”, a 1:1 mixture of NCeONP and ZrO₂ at 100 μm;

FIG. 16 is a titer plot of the mAb Titer (g/L) versus days showing an increase in concentration of the 1:1 mixture of NCeONP and ZrO₂ nanoparticles;

FIG. 17 shows that the glycosylation patterns or relative abundance of each glycan species remains the same as between the control and the indicated concentrations of 100 micromolar, 500 micromolar and 1000 micromolar;

FIG. 18A provides a plot of cell growth versus concentration for NCeONP;

FIG. 18B provide a plot of cell growth versus concentration for a 1:1 mixture of NCeONP and ZrO₂ nanoparticles (NP);

FIG. 18C provides a plot of cell growth versus concentration for ZrO₂ NP;

FIG. 19A provides a plot of cell growth versus concentration of NCeONP;

FIG. 19B provides a plot of cell growth versus concentration for a 1:1 mixture of NCeONP and ZrO₂ NP;

FIG. 19C provides a plot of cell growth versus concentration for ZrO₂; and

FIG. 20 is a plot of dissolved oxygen versus time for a 3.0 mm size ethylene 1-octene copolymer bead containing a 1:1 nanoporous cerium oxide nanoparticle macrostructures at a 10.0% (wt.) loading in the bead in the presence of hydrogen peroxide.

### DETAILED DESCRIPTION

The present invention stands directed at cerium oxide nanoparticles and/or nanoporous cerium oxide nanoparticle macro-structures and/or zirconium dioxide nanoparticles which produce oxygen upon exposure to reactive oxygen species (ROS) and/or scavenge free radicals. Accordingly, wherever reactive oxygen species may be produced, such as in cell culture media, chloroplasts or algae, one can now utilize additives such as cerium oxide nanoparticles, nanoporous cerium oxide nanoparticle macro-structures and/or zirconium dioxide nanoparticles to convert the reactive oxygen species to oxygen and/or scavenge the presence of any free radicals, particularly those made during cell metabolism that may otherwise build-up in cells and cause damage.

In particular, the cerium oxide nanoparticles and/or nanoporous cerium oxide nanoparticle macro-structures and/or zirconium dioxide nanoparticles produce oxygen from reactive oxygen species produced by cell culture mediums, such as hydrogen peroxide produced by cells under environmental stress. Reactive oxygen species (ROS) such as superoxide anion (O₂), hydrogen peroxide (H₂O₂) and hydroxy radical (HO·) are understood herein to comprise non-radical oxygen species and radical oxygen species formed by partial reduction of oxygen. By way of further example, reactive oxygen species is therefore reference to oxygen hydroxyl radicals (·OH), hydroxyl ion (OH⁻) superoxide anion radical (·O₂⁻), hydrogen peroxide (H₂O₂), oxygen (O₂), or superoxide anion (O₂^{·-}). The cerium oxide nanoparticles, nanoporous cerium oxide nanoparticle macro-structures, and/or zirconium dioxide nanoparticles may be used directly in particulate form within a bioreactor containing a biological cell culture medium to produce oxygen from reactive oxygen species and/or scavenge the presence of free-radicals, or may be loaded into a polymer film which then can be used in the bioreactor, or the film itself may be utilized to form the bioreactor to contain the cell culture medium. Such bioreactor may preferably have a size in the range of 100 ml to 100 liters. The cell culture mediums herein then include but are not limited to cells sourced from animal or plant sources. Cells are therefore contemplated to include mammalian cells, bacterial cells, yeast cells, algae cells, and insect cells. Accordingly, a bioreactor herein is understood as any container that serves to contain a cell culture medium along with cells for biomanufacturing and/or cellular growth/expansion. Cellular growth/expansion herein is reference to an increase in the mass of a cell and/or cell proliferation which refers to an increase in the number of cells. Biomanufacturing herein is reference to the production of biological products from living cells.

When used in particulate form within a cell culture medium, the level of any one of the cerium oxide nanoparticles, nanoporous cerium oxide nanoparticle macro-structures, or zirconium dioxide nanoparticles preferably falls in the range of 1.0 nanomolar (1.0 nM) to 1000 millimolar (1000 mM). More preferably, the range is from 1.0 nM to 100 mM, and even more preferably, the range is from 1.0 nM to 1 mM. In addition, when two or more of said cerium oxide nanoparticles, nanoporous cerium oxide nanoparticle macro-structures and zirconium dioxide nanoparticles are present, the combined level is also preferably in the range of 1.0 nM to 1000 mM, and even more preferably, the range is from 1.0 nM to 100 mM, or 1.0 nM to 1 mM. Accordingly, if cerium oxide nanoparticles, nanoporous cerium oxide nanoparticle macro-structures and zirconium dioxide nanoparticles are all present in a bioreactor, the combined level is again preferably in the range of 1.0 nM to 1000 mM, or 1.0 nM to 100 mM, or 1.0 nM to 1 mM. The cerium oxide nanoparticles, nanoporous cerium oxide nanoparticle macro-structures and/or zirconium dioxide nanoparticles can therefore be premixed with the cell culture medium or can be added to the cell culture medium in the bioreactor at selected intervals.

The cerium oxide nanoparticles, nanoporous cerium oxide nanoparticle macro-structures and zirconium dioxide nanoparticle additives may therefore be provided in particulate form and may also be provided as encapsulated within a polymeric bead. Such polymeric beads may preferably have a size in the range of 10 micrometer to 10 millimeter diameter and are preferably spherical but there can also be non spherical forms such as cylindrical, and therefore may be configured to contain the indicated additives as part of a porous core/shell type structure. In addition, the nanoporous cerium oxide nanoparticles, nanoporous cerium oxide nanoparticle macro-structures and zirconium dioxide nanoparticles, are contemplated as being provided in the form of a micelle, i.e. colloidal particles with a hydrophobic core and a hydrophilic shell.

When the cerium oxide nanoparticles, nanoporous cerium oxide nanoparticle macro-structures, and/or zirconium dioxide nanoparticles are each on their own loaded as an additive into a polymeric film, that may be used within the bioreactor, or even to form the bioreactor itself, the loading level in the film of the individual additive on its own is preferably in the range of 0.01 wt. % to 10.0 wt. %, more preferably 0.01 wt. % to 5.0 wt. %. Accordingly, one may preferably utilize 0.01 wt. % to 10.0 wt. % of cerium oxide nanoparticles, or 0.01 wt. % to 10.0 wt. % of nanoporous cerium oxide nanoparticle macro-structures, or 0.01 wt. % to 10.0 wt. % of zirconium dioxide nanoparticles in a polymer film that may be used within the bioreactor or as the polymeric film that forms the bioreactor. In addition, when two or more of the cerium oxide nanoparticles, nanoporous cerium oxide nanoparticle macro-structures and zirconium dioxide nanoparticles are present in the polymer film, the combined loading level is also preferably in the range of 0.01 wt. % to 10.0 wt. %. Accordingly, if cerium oxide nanoparticles, nanoporous cerium oxide nanoparticle macro-structures and zirconium dioxide nanoparticles are all present in the polymer film, the combined loading is also preferably in the range of 0.01 wt. % to 10.0 wt. %.

As may therefore be appreciated, the polymer film containing the cerium oxide nanoparticles and/or nanoporous cerium oxide nanoparticle macro-structures and/or zirconium dioxide nanoparticles, or any combination thereof, can physically serve as the bioreactor to contain the biological cell culture. The films therefore containing such cerium oxide nanoparticles and/or nanoporous cerium oxide nanoparticle macro-structures and/or zirconium dioxide nanoparticles can similarly produce oxygen from reactive oxygen species and/or scavenge the free radicals that are produced by the cells and released into the cell culture media within the bioreactor container.

Reference to cerium oxide nanoparticles is reference to nanoparticles (NP) preferably having a diameter (largest linear dimension) in the range of 10.0 nm to 100.0 nm, including all individual values and increments therein. Accordingly, the cerium oxide nanoparticles employed herein preferably have a diameter in the range of 10.0 nm to 50.0 nm or 10.0 nm to 30.0 nm or 20.0 nm to 30.0 nm. The zirconium dioxide nanoparticles preferably have a diameter in the range of 10.0 nm to 2000.0 nm. More preferably, the zirconium dioxide particles have a diameter in the range of 10.0 nm to 100.0 nm, or 10.0 nm to 50.0 nm, or 10.0 nm to 20.0 nm, or 10.0 nm to 15.0 nm.

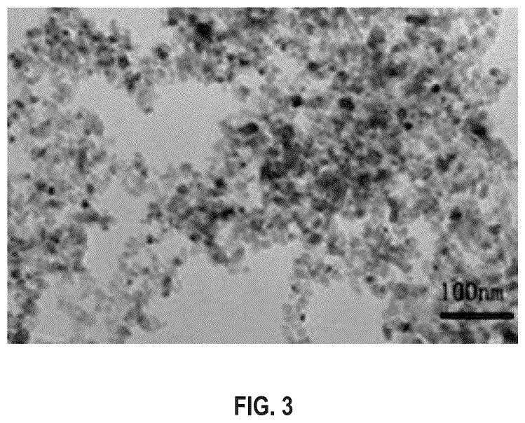

Reference to a nanoporous cerium oxide nanoparticle macro-structure is reference to the feature that a plurality of the particles associate or adhere to one another where the macro-structure has its own pore size diameter. The above referenced cerium oxide nanoparticles are then preferably degassed with nitrogen for a preferred period of 30 minutes to 60 minutes. This is then preferably followed by heating at elevated temperature, and preferably at the temperature range of 50° C. to 900° C. for a preferred period of 1.0 hour to 3.0 hours, more preferably 1.0 hour to 2.0 hours. Accordingly, such heating of the cerium oxide nanoparticles was observed to form a plurality of nanoporous cerium oxide nanoparticle macro-structures 12 illustrated in FIG. 2 having macro-structure pores 14.

The macro-structure pores 14 that are formed by the cerium oxide nanoparticle macro-structure 12 preferably have a diameter (largest linear dimension) as indicated by arrow 15 in the range of 10 nm to 1100 nm, more preferably, 10 nm to 750 nm or 10 nm to 500 nm or 10 nm to 250 nm or 10 nm to 100 nm or 10 nm to 50 nm or 10 nm to 25 nm. In addition, the nanoporous cerium oxide nanoparticle macro-structures 12 themselves are contemplated to have a preferred diameter (largest linear dimension) as indicated by arrow 16 in the range of 50 nm to 30,000 nm.

In one particularly preferred embodiment, the nanoporous cerium oxide nanoparticle macro-structures that are formed (herein abbreviated as NCeONP or NCeONP macro-structures) have a binary size distribution with respect to both their macro-structure diameter 16 and macro-structure pore diameter 15. A binary size distribution is reference to two distributions of size ranges for both the macro-structure diameter and macro-structure pore diameter. That is, the preparation methods herein preferably provide a nanoporous cerium oxide nanoparticle macro-structure that has the following binary size distribution: (1) macro-structure diameter in the range of 10 nm to 300 nm with a macro-structure pore diameter in the range of 5 nm to 30 nm, more preferably 10 nm to 20 nm; and (2) macro-structure diameter in the range of 5,000 nm to 30,000 nm with a macro-structure pore diameter in the range of 900 nm to 1100 nm.

In another particularly preferred embodiment, one may employ a 1:1 mixture of cerium oxide nanoparticles with zirconium dioxide nanoparticles. However, in the broad context of the present disclosure this ratio can fall in the range of 1:10 to 10:1. Similarly, in preferred embodiment, one may employ a 1:1 mixture of cerium oxide nanoparticles with zirconium dioxide nanoparticles. Moreover, in the broad context of the present disclosure, this ratio of the nanoporous cerium oxide nanoparticle macro-structures to the zirconium dioxide nanoparticles can similarly fall in the range of 1:10 to 10:1, and in in one particularly preferred embodiment, the ratio is a 1:1 mixture of nanoporous cerium oxide nanoparticle macro-structures to zirconium oxide nanoparticles. It is therefore understood that the bioreactor herein provides these aforementioned preferred levels of cerium oxide nanoparticles to zirconium dioxide nanoparticles as well as the preferred levels of nanoporous cerium oxide nanoparticle macro-structures to zirconium dioxide nanoparticles.

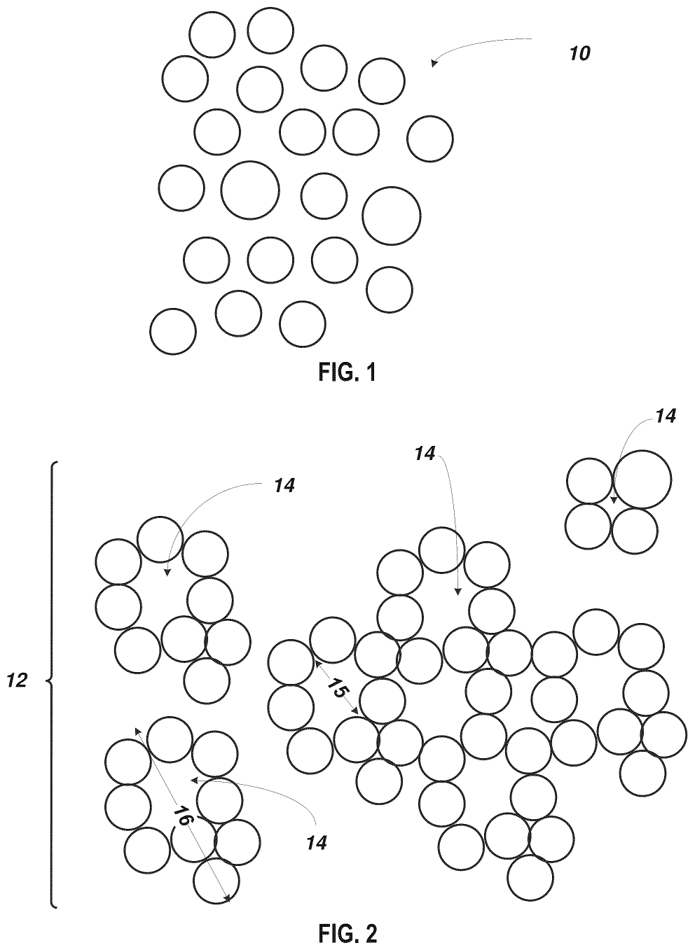

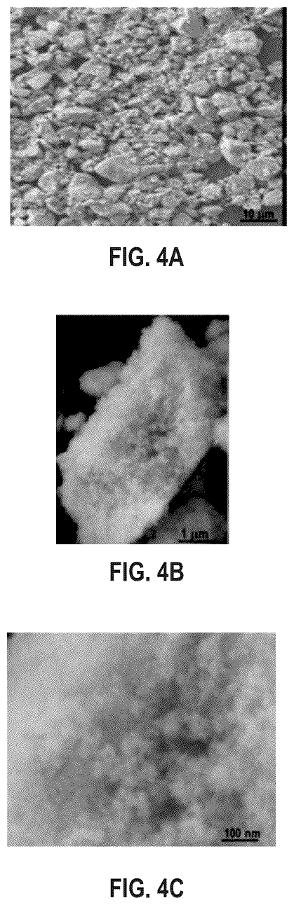

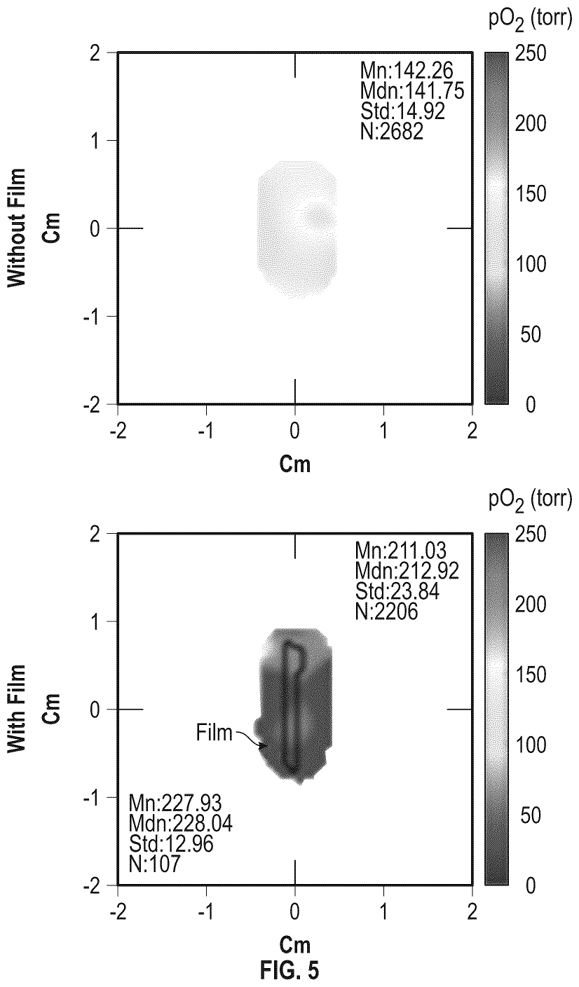

FIG. 3 is a scanning electron micrograph of the cerium oxide nanoparticles employed herein to form the nanoporous cerium oxide nanoparticle macro-structure. As noted above, such starting cerium oxide nanoparticles preferably had a diameter of 20 nm to 30 nm. FIGS. 4A, 4B and 4C, respectively, provide scanning electron micrographs at increasing magnification showing the nanoporous cerium oxide nanoparticle macro-structure herein formed from the cerium oxide nanoparticles of FIG. 3, wherein the macro-structure itself forms macro-structure pores 14 (see again FIG. 1).

The cerium oxide nanoparticles and/or the nanoporous cerium oxide nanoparticle macro-structures (NCeONP) and/or the zirconium dioxide nanoparticles may therefore preferably be incorporated into a polymer film, which film is then preferably for use to contact or contain biological cellular media where the polymer film is capable of: (1) converting reactive oxygen species such as hydrogen peroxide to oxygen to mitigate oxidative stress on the cells and to increase cell viability; and/or (2) scavenge free radicals. The polymer film is contemplated to include both thermoplastic and thermoset type polymer film. Preferably, the polymer film includes poly(vinyl chloride), and in particular, polyvinyl chloride film that does not contain any plasticizers, and in particular, does not contain phthalate plasticizers. The polymer film may also include polyolefin films, including polyethylene and polypropylene film as well as ethylene-propylene copolymers. Other polymer films may include polyethylene-co-vinyl acetate films, polyester films, polyamide films, cellulosic films, and polyurethane films. These polymer films can be single layer or multiple layers with the layer that is contact with the fluid embedded or coated with nanoparticles. Such polymer films may therefore be preferably employed to form the bioreactor and the films may be single layer films or multilayer films where the film loaded with cerium oxide nanoparticles and/or the nanoporous cerium oxide nanoparticle macro-structures and/or zirconium dioxide nanoparticles serve as the internal or contact layer in contact with the cell culture medium. The polymer films also preferably have a thickness in the range of 0.01 mm to 10.0 mm, including all values and increments therein.

As alluded to above, the polymer films (whether placed within a bioreactor or to form the bioreactor) preferably have an individual loading level of cerium oxide nanoparticles or the nanoporous cerium oxide nanoparticle macro-structures or zirconium dioxide nanoparticles at a level in the range of 0.01 wt. % to 10.0 wt. %, including all values and increments therein. In addition, the cerium oxide nanoparticles, nanoporous cerium oxide nanoparticle macro-structures and zirconium dioxide nanoparticles may be selectively mixed and similarly be preferably present in the polymer film at a combined loading level of 0.01 wt. % to 10.0 wt. %. In other words, as mixed together, two or more of the cerium oxide nanoparticles or the nanoporous cerium oxide nanoparticle macro-structures or zirconium dioxide nanoparticles are preferably present at a combined loading level that totals 0.01 wt. % to 10.0 wt. %. For example, when the load level is 10.0 wt. %, the level of cerium oxide nanoparticles may preferably be 3.0 wt. %, the level of cerium oxide macro-structures may be 5.0 wt. % and the level of zirconium dioxide nanoparticles may be 2.0 wt. %.

Expanding upon the above, it can now be appreciated that the zirconium dioxide nanoparticles may replace a portion of the cerium oxide nanoparticles or nanoporous cerium oxide nanoparticle macro-structures. In the preferred case of film loadings, and by way of further example, the zirconium dioxide nanoparticles may therefore preferably be present with the cerium oxide nanoparticles and/or cerium oxide macro-structures at a level of up to 9.9 wt. % to 0 wt. %. For example, the loading level of zirconium dioxide nanoparticles in the polymer film may preferably be 9.9 wt. % and the level of cerium oxide nanoparticles and/or cerium oxide macro-structures is then 0.1 wt. %, so the total loading level in the polymer film is 10.0 wt. %. Or, in another example, the level of cerium oxide nanoparticles and/or nanoporous cerium oxide macro-structures in a polymer film may be at a level of 5.0 wt. % and the level of zirconium dioxide nanoparticles may be in the range of 0.1 wt. % to 5.0 wt. %. In yet another example, the level of cerium oxide nanoparticles and/or nanoporous cerium oxide macro-structures in a polymer film may be 7.0 wt. % and the level of zirconium dioxide nanoparticles may be in the range of 0.1 wt. % to 3.0 wt. %.

The polymer films (without any loaded amounts of cerium oxide nanoparticles or nanoporous cerium oxide nanoparticle macro-structures or zirconium dioxide) may also preferably be coated with the cerium oxide nanoparticles and/or the nanoporous cerium oxide nanoparticle macro-structures (NCeONP) and/or the zirconium dioxide nanoparticles. Such coated films may therefore serve as the internal surface of the bioreactor, namely that surface that is in contact with a given cell culture medium which can be referred to as the contact layer. Such coating may be achieved by preparing formulations containing the cerium oxide nanoparticles and/or the nanoporous cerium oxide nanoparticle macro-structures (NCeONP) and/or the zirconium dioxide nanoparticles along with coating polymers in solvents followed by coating of the formulation on the surface of the film and allowing it to dry. It is contemplated that such coating polymers preferably comprise thermoplastic polymers that are soluble in selected solvent(s) that are suitable for coating purposes. The loading of coating polymer in the solvent may preferably be in the range of 0.01 wt. % to 10.0 wt. % and the loading level of any one of the cerium oxide nanoparticles or the nanoporous cerium oxide nanoparticle macro-structures (NCeONP) or the zirconium dioxide nanoparticles in the solvent may therefore be in the range of 0.01 wt. % to 10.0 wt. %. Moreover, the loading level of any two of the cerium oxide nanoparticles and/or the nanoporous cerium oxide nanoparticle macro-structures (NCeONP) and/or the zirconium dioxide nanoparticles in the solvent may preferably be in the range of 0.01 wt. % to 10.0 wt. %. In addition, the loading level of all three of the cerium oxide nanoparticles and/or the nanoporous cerium oxide nanoparticle macro-structures (NCeONP) and the zirconium dioxide nanoparticles in the solvent may also be in the range of 0.01 wt. % to 10.0 wt. %. The coating thickness is preferably in the range of 0.01 microns to 1000 microns. Such coatings can also be applied on non-polymeric surfaces like stainless steel or metallic reactor surfaces.

As noted, the polymer films herein, containing cerium oxide nanoparticles and/or nanoporous cerium oxide nanoparticle macro-structures and/or zirconium dioxide nanoparticles, when in contact with cells, are contemplated to increase cell viability by producing oxygen from reactive oxygen species such as hydrogen peroxide and/or scavenge free-radicals. The polymer films herein may therefore be configured as a bioreactor to contain cells in liquid cell culture media which cells are employed to produce biological products and/or undergo cellular growth/expansion, wherein reactive oxygen species such as hydrogen peroxide present or produced in the liquid media can be converted to oxygen and/or the films containing cerium oxide nanoparticles and/or nanoporous cerium oxide nanoparticle macro-structures and/or zirconium dioxide nanoparticles scavenge free radicals. As noted, the films may therefore preferably be in the form of a bioreactor vessel or container. The polymer films forming the bioreactor are therefore contemplated to reduce the environmental stress on the cells due to the presence or production of, e.g., hydrogen peroxide within the container and increase cell viability and cellular growth/expansion. This is particularly contemplated to improve biomanufacturing and the production of biologic products from living cells and the expansion/growth of cells.

The cells that are contemplated herein that may be employed for biomanufacturing, and which are now exposed to cerium oxide nanoparticles and/or cerium oxide macro-structures and/or zirconium dioxide nanoparticles, or to films containing such components, can operate with mitigation in oxidative stress to increase their cell viability, include recombinant E. coli, yeast cells or animal cells, i.e., Chinese hamster ovary (CHO) cells or hybridoma cells. E. coli is the microbial system of choice for the expression of heterologous proteins. More preferably, the cells that are contemplated herein for biomanufacturing with reduced oxidative stress include CHO cells, human embryonic kidney (HEK) cells, cells derived from non-secreting murine myeloma (NSO cells), neuronal cells, baby hamster kidney fibroblasts (BHK) cells. The biologically-produced products herein produced from such cells with reduced oxidative stress are contemplated to include hormones, blood products, cytokines, growth factors, genes, fusion proteins, insulin, interferon, and monoclonoal antibody (mAb) products. The biologically-produced products may also include therapeutic proteins, peptides, polysaccharides, vaccines, industrial chemicals, antibiotics, food and agricultural products, chemicals, and polymers. Accordingly, the biologically-produced products may include drugs, therapeutics, biologics, and biosimilars. The biologically-produced products may also include those obtained from fermentation, biofuels, micro algae and products from micro-algae and cultured meat. In addition, the cells herein that are contemplated for cellular growth/expansion are contemplated to include those used in cell therapy. For example, these cells refer to those used in the transfer of new cells, or cells that have been modified in a laboratory to achieve particular characteristics in the body to prevent or treat a disease. Examples of such cells include stem cells, CAR T-Cells, Natural killer cells (NK cells), T cells, also called as T lymphocytes and thymocytes.

As alluded to above, it is contemplated that cerium oxide nanoparticles and/or the nanoporous cerium oxide nanoparticle macro-structures and/or zirconium dioxide nanoparticles, operate as noted to convert reactive oxygen species such as hydrogen peroxide to oxygen or scavenge free radicals. As can now be appreciated, the aforementioned additives may therefore be used directly in the cell culture mediums to achieve such mitigation in reactive oxygen species or as loaded into a polymer film that is in contact with the cell culture medium, or as a coating on the polymer film that is contact with the cell culture medium. It is contemplated that this is the result of the following reaction occurring, due to the presence of the cerium oxide nanoparticles and/or nanoporous cerium oxide nanoparticle macro-structures, upon exposure to the particular presence of hydrogen peroxide:

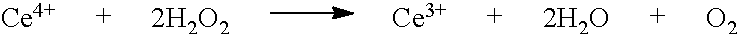

### Working Examples

Example 1: Polymer films were prepared from plasticized poly(vinyl chloride) that contained 5.0 wt. % of nanoporous cerium oxide nanoparticle macro-structures. The plasticizer was DEHT which is considered as a non-toxic alternative to DEHP currently used in blood bags. Polymer films were also prepared from polyethylene that contained 2.0 wt. % of a 1:1 mixture of nanoporous cerium oxide nanoparticle macro-structures in combination with zirconium dioxide nanoparticles. Preparation of ceria compounded PVC-DEHT films proceed as follows.

One can directly mix the nanoporous cerium oxide nanoparticle macro-structures with the molten PVC to extrude or compression mold into nanocomposite films preferably using a twin screw extruder (Equipment: Feeder-Brabender; Grinder-BPM Grinder; Water bath at room temperature; Twin-screw extruder-Welex®). Screw configuration, feed rates of the PVC and NCeONP, and screw speed control the rates of shear and elongation applied to the nanocomposite and the residence time in the extruder. The nanoporous cerium oxide nanoparticle macrostructure-PVC composite sheets with two concentrations of nanoporous cerium oxide nanoparticle macro-structures were prepared, namely one at 2.0 wt. % and one at 5.0 wt. %. Using this approach, one obtains more than 30 sheets, (4×5 inches) with 0.10 cm thickness (1-5 wt % NCeONP loading). The following extruder parameters were used: Speed—95, Melt temp—150° C.; Melt pressure 70 PSI, and Load—32 wt %. A similar processing approach was used for preparation of PE-XNA composite films.

Such films were treated with a hydrogen peroxide solution and after a period of 24 hours, a color change was observed, confirming the conversion of hydrogen peroxide to water and oxygen. The color change indicates that oxygen generation is accompanied by the conversion of pale yellow Ce+4 to brownish Ce+3.

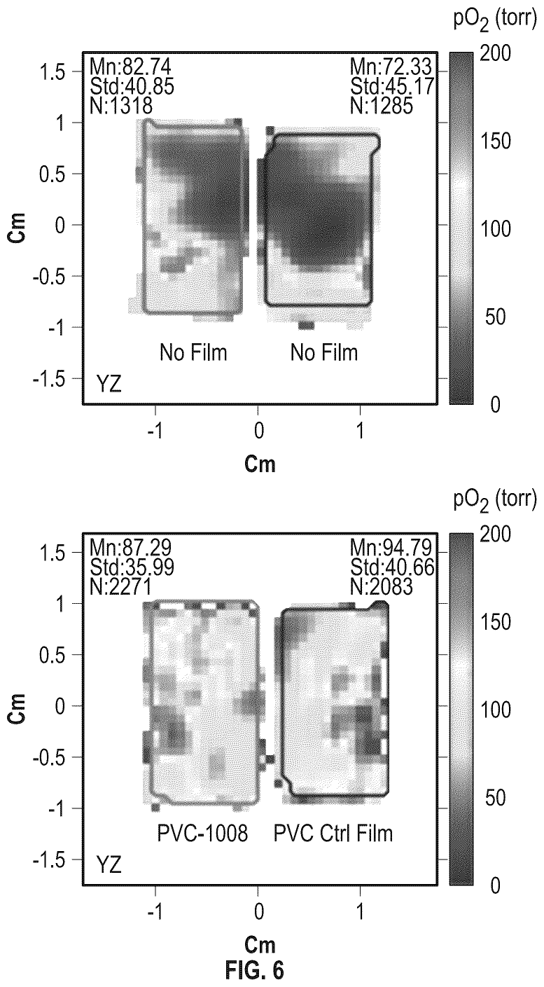

Example 2: This example demonstrates the increase in oxygen levels in media that contains hydrogen peroxide as the representative reactive oxygen hydrogen species. More specifically, plasticized poly(vinyl chloride) that contained 5.0 wt. % of nanoporous cerium oxide nanoparticle macro-structures was employed which was placed in a bottle of deionized water containing 3.0% H₂O₂ and 0.5 mM of trityl (triphenylmethyl chloride). Inversion recovery electron spin echo (IRESE) image sequencing was conducted after a period of 48 hours and the results are presented in FIG. 5 against a control (bottle containing 3.0% H₂O₂ and 0.5 mM of trityl in the absence of any film). As can be seen the bottle without the film indicated an average partial pressure of oxygen of 142.26+/−0.29 torr. The bottle containing the aforementioned level of H₂O₂ along with the plasticized poly(vinyl chloride) film loaded with 5.0 wt. % of nanoporous cerium oxide nanoparticle macro-structures indicated an average partial pressure of oxygen of 211.03+/−0.51 torr. This confirms that polymeric film, containing the nanoporous cerium oxide nanoparticle macro-structures, could convert the H₂O₂ to oxygen.

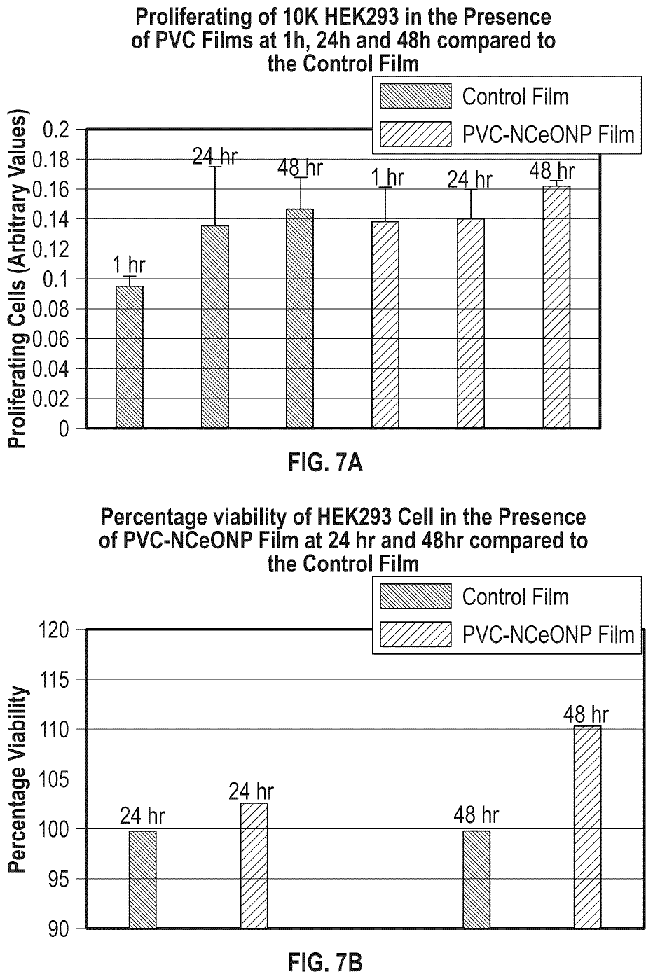

Example 3. This example is similar to Example 2, where the bottle containing the 3.0% H₂O₂ and 0.5 mM of trityl now included human embryonic kidney or HEK293 cells. FIG. 6 provides the oxygen mapping of the cells in the media both with and without the plasticized poly(vinyl chloride) that contained 5.0 wt. % of nanoporous cerium oxide nanoparticle macro-structures. The partial pressure of oxygen observed for the cell media contain 3.0% H₂O₂ and 0.5 mM trityl, after 72 hours, fell in the range of 0-100 torr. The partial pressure of oxygen for the cell media containing 3.0% H₂O₂ and 0.5 mM trityl, in the presence of unplasticized PVC film containing 5.0 wt. % of nanoporous cerium oxide nanoparticle macro-structures, fell in the range of 100-200 torr.

Example 4. In this example, polymer films were again prepared from plasticized poly(vinyl chloride) that contained 5.0 wt. % of nanoporous cerium oxide nanoparticle macro-structures. Polymer films were also prepared from polyethylene that contained 2.0 wt. % of a 1:1 mixture of nanoporous cerium oxide nanoparticle macro-structures in combination with zirconium dioxide nanoparticles. The films were punched into 4.0 mm disks using a hollow puncher. A stack of 3 disks were used for each samples of control as well as test samples.

The HEK293 cells were plated in 96-wells one day before placing the appropriate 3-disk stacked films in the well. The next day, cultural media was replaced with fresh media, and the 3 disk stacked film was placed. At the end of the experiment, films and spent media were removed, and MTT solution was added and incubated at 37° C. BOD incubator for 3 h. Precipitated formazan was dissolved in DMSO and shakedown the rocker for 15 mins. The reading was taken at 590 nm on BioTek (Synergy LX) multimode plate reader.

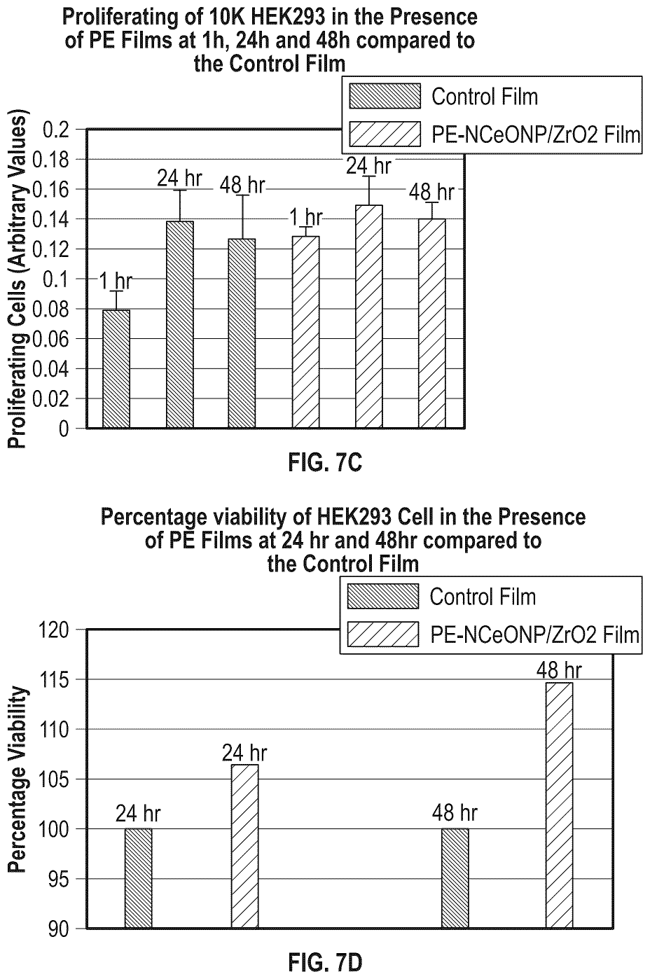

Reference is made to FIG. 7A which is a plot of the proliferating cells (arbitrary values) of 10K HEK 293 in the presence of plasticized PVC films containing 5.0 wt. % nanoporous cerium oxide nanoparticle macro-structures at 1 hr, 24 hr and 48 hr. as compared to a control film (plasticized PVC film without any nanoporous cerium oxide nanoparticle macro-structures). Proliferating cells is reference to increased cell growth in terms of number of cells and was determined by MTT assy. As can be seen, the level of proliferating cells trended upward at 1 hr, 24 hr and 48 hr when the cells were exposed to plasticized PVC film containing 5.0 wt. % of nanoporous cerium oxide nanoparticle macro-structures.

Reference is made to FIG. 7B, which is a plot of percentage viability at 24 hrs. for the control plasticized PVC film and the plasticized PVC film containing 5.0 wt. % nanoporous cerium oxide nanoparticle macro-structures. Percentage viability is reference to the feature of assigning 100% cell viability to a control and the percent viability is any increase beyond 100% which is then considered an enhancement in percentage viability and is determined by counting the number of cells using MTT assay. As can be seen, after 24 hours and 48 hours, the percentage cell viability was enhanced with respect to those cells in contact with films containing 5.0 wt. % nanoporous cerium oxide nanoparticle macro-structures.

Reference is made to FIG. 7C which is a plot of the proliferating cells (arbitrary values) of 10K HEK 293 in the presence of polyethylene film containing 2.0 wt. % nanoporous cerium oxide nanoparticle macro-structures and zirconium dioxide nanoparticles at a 1:1 ratio, at 1 hr, 24 hr and 48 hr. as compared to a control film (polyethylene) without any nanoporous cerium oxide nanoparticle macro-structures and zirconium dioxide nanoparticles. Proliferating cells is reference to increased cell growth in terms of number of cells and was determined by MTT assy. As can be seen, the level of proliferating cells trended upward at 1 hr, 24 hr and 48 hr when the cells were exposed to polyethylene film containing 2.0 wt. % nanoporous cerium oxide nanoparticle macro-structures and zirconium dioxide nanoparticles at a 1:1 ratio.

Reference is made to FIG. 7D which is a plot of percentage viability at 24 hrs. for the control polyethylene film and the polyethylene film containing 2.0 wt. % nanoporous cerium oxide nanoparticle macro-structures and zirconium dioxide nanoparticles at a 1:1 ratio. As can be seen, after 24 hours and 48 hours, the percentage cell viability was enhanced with respect to those cells in contact with films containing 2.0 wt. % nanoporous cerium oxide nanoparticle macro-structures in combination with zirconium dioxide nanoparticles at a 1:1 ratio.]

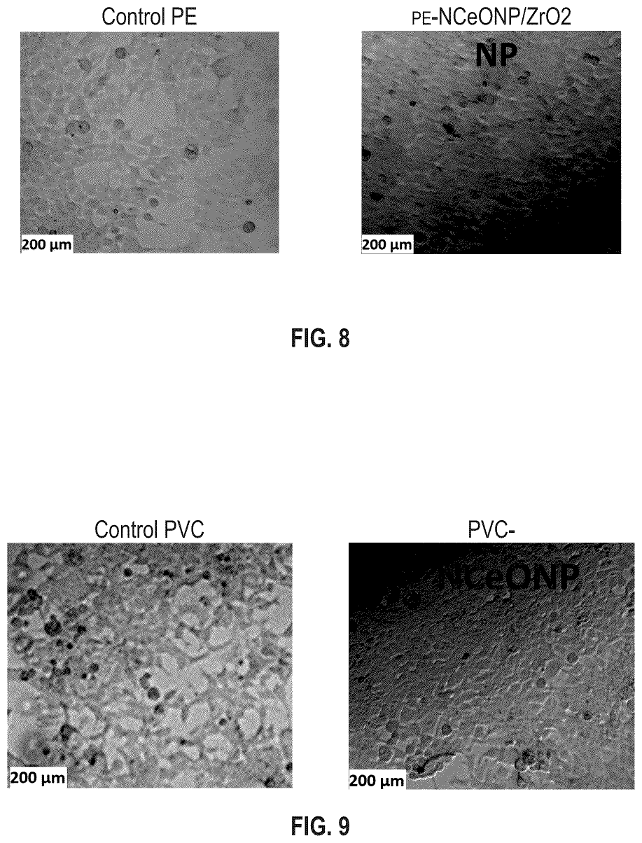

FIG. 8 illustrates the morphology of the HEK cells after 48 hours in the presence of plasticized PVC films containing 5.0 wt. % nanoporous cerium oxide nanoparticle macro-structures) versus a control (plasticized PVC film). More viable cells were observed in the plasticized PVC films containing 5.0 wt. % nanoporous cerium oxide macrostructures than compared to plasticized films without such additive.

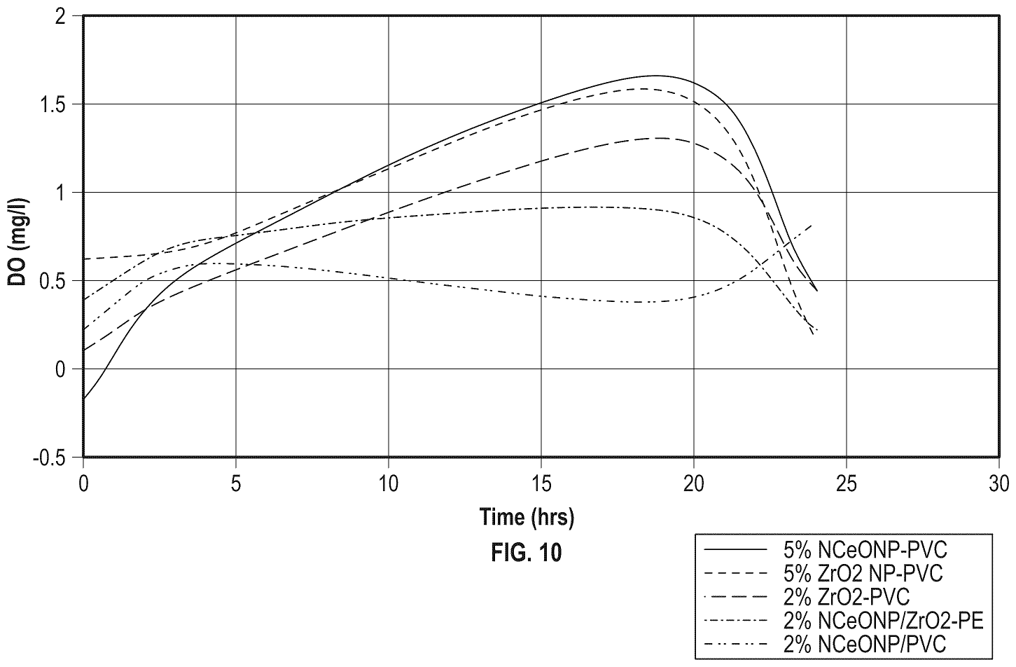

FIG. 9 illustrates the morphology of the HEK cells after 48 hours in the presence of polyethylene film containing 2.0 wt. % nanoporous cerium oxide nanoparticle macro-structures and zirconium dioxide nanoparticles at a 1:1 ratio. More viable cells were observed in the polyethylene film containing 2.0 wt. % nanoporous cerium oxide nanoparticle macro-structures and zirconium dioxide nanoparticles at a 1:1 ratio than in the control polyethylene film.

### Additional Working Examples

Example 1B: The goal of this example was to compare different nanocomposite films, namely NCeONP-PVC and ZrO₂ NP-PVC at 2 different loadings (2.0 wt. % and 5.0 wt. %) and a 1:1 mixture of NCeONP: ZrO₂ NP at 2.0 wt. % loading in PE along with a consideration of their ability to produce oxygen at equal weight of the film under identical conditions and concentration of H₂O₂. The typical procedure used is as follows:

Add 90 mL of DI water to a clean and dry flask. Add 4 g of each type of film into the flask and close the lid before shaking well. Immediately add 10 mL 3% H₂O₂ to the flask, shake well, and leave still for 5 minutes. Take the first measurement using the dissolved oxygen DO) meter and record the time. This will be the 0 value for DO and time. Wait for 3.5-4 hours before taking the next measurement. Shake the flask again, wait for 5 minutes, and take DO and time measurements. Repeat the process for this specific vial and any subsequent flasks until 5, 10, 15, 20, 24, 48-hour measurements have been taken. Subtract the residual DO readings that exist just with H₂O and H₂O₂ from each of the readings with the films. The following can be inferred.

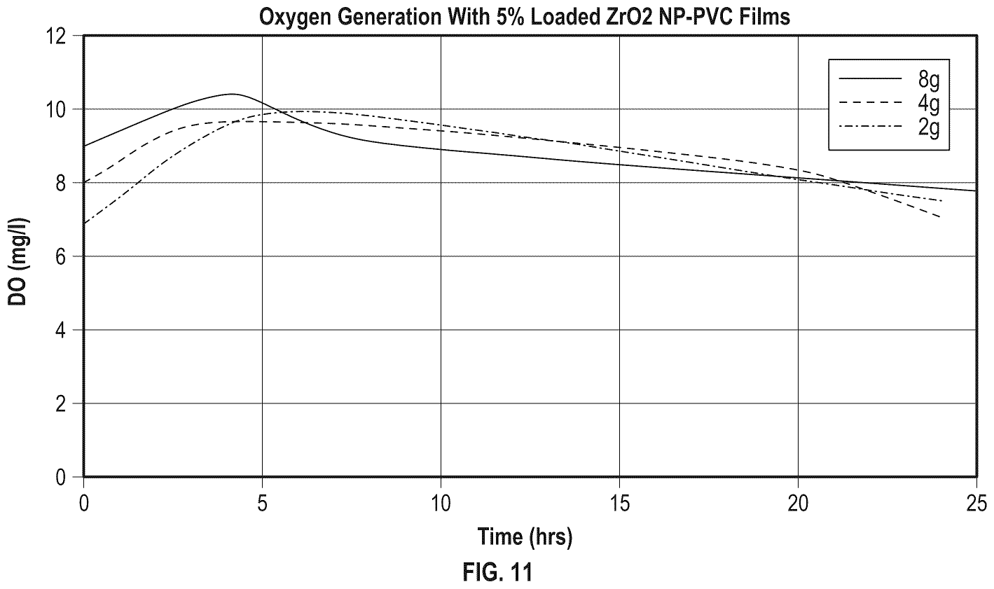

Looking at FIG. 10, which is a plot of dissolved oxygen (DO) versus time for the compared films, the peak oxygen generation is reached at about 20 hrs, with 5.0 wt. % loaded PVC films producing the highest oxygen compared to 2.0 wt. % loaded PVC films. The 2.0 wt, % ZrO₂ NP-PVC generates higher DO among the 2.0 wt. % loaded films. Surprisingly, the 2% loaded NCeONP-PVC film shows the relative lowest DO, unlike the corresponding 5% loaded series.

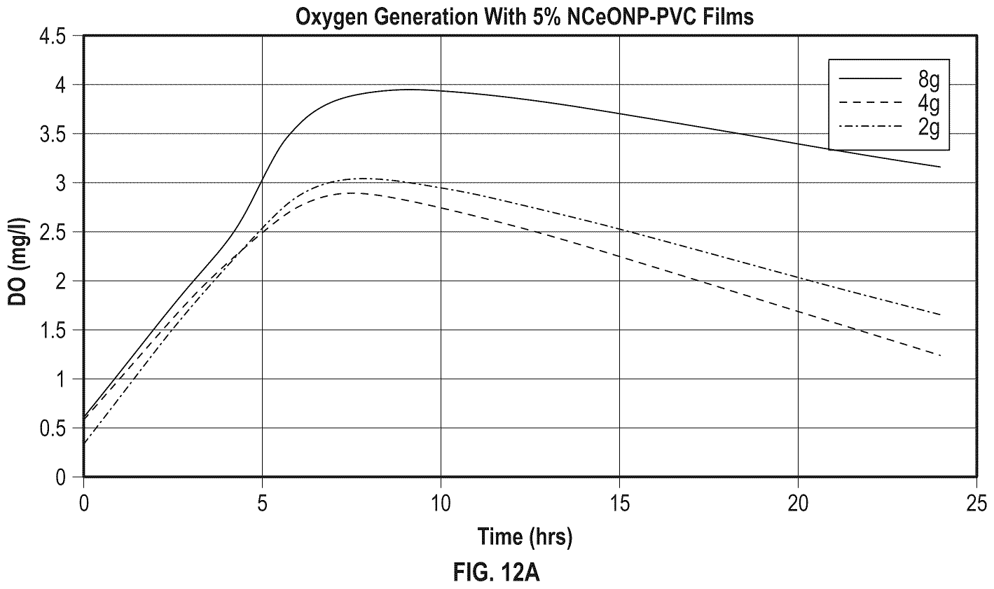

Example 2B: The following example was conducted similarly to Example 1 but focused on 5.0 wt. % ZrO₂ NP-PVC film's ability to generate DO and their concentration/weight dependency. As can be seen in FIG. 11, the DO generation increases with an increase in the weight of the film (2 g, 4 g, and 8 g) used under identical experimental conditions. This indicates that one can control the DO generation through loading of zirconium dioxide nanoparticles in the nanocomposite films.

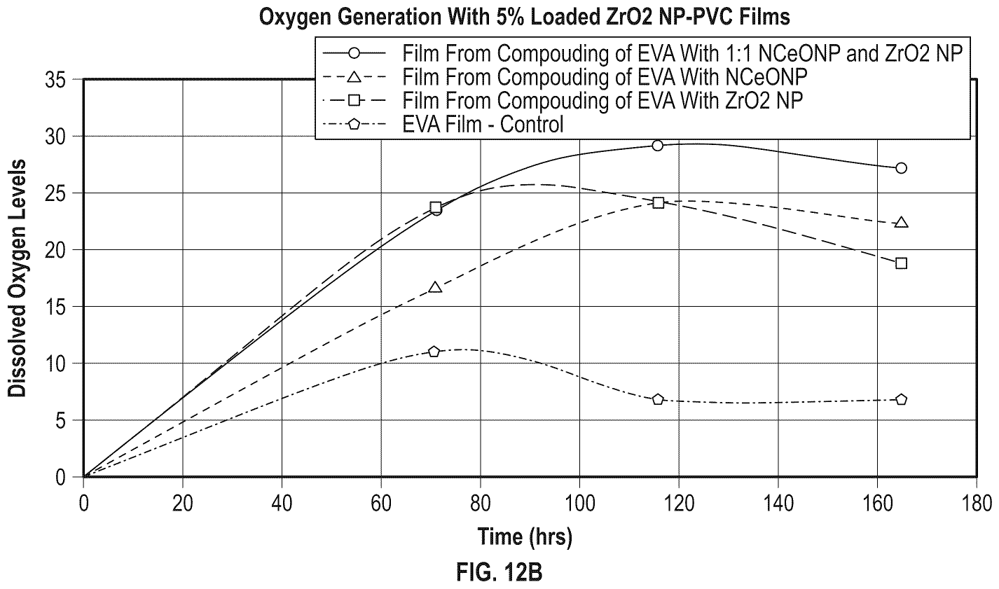

Example 3B: The following example was conducted similarly to Example 2 but focused on 5.0% wt. NCeONP-PVC film's ability to generate DO and their concentration/weight dependency. As can be seen from FIG. 12A, the DO generation increases with an increase in the weight of the film (2 g, 4 g, and 8 g) used under identical experimental conditions. FIG. 12B is a plot of dissolved oxygen (DO) versus time shown DO generation by ethylene vinyl acetate (EVA) compounded with the various indicated additives.

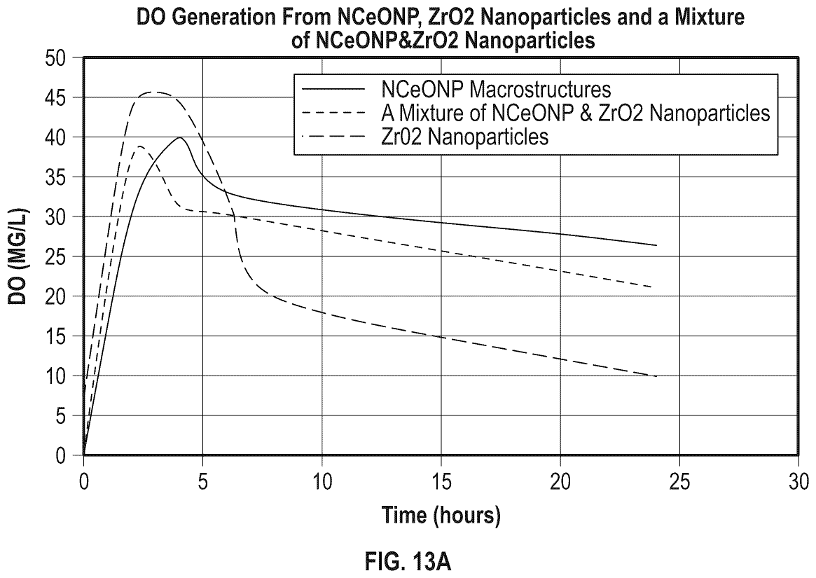

Example 4B. The following example was conducted to evaluate the ability of nanoporous cerium oxide nanoparticle macro-structures, zirconium dioxide nanoparticles, and mixtures of nanoporous cerium oxide nanoparticle macro-structures mixed with zirconium dioxide nanoparticles, in particulate form, to generate oxygen in the presence of a reactive oxygen species, namely hydrogen peroxide. As can be seen in FIG. 13A, when in particulate form, distilled water containing hydrogen peroxide at a concentration of 0.3%, a particulate form of nanoporous cerium oxide nanoparticle macro-structures at a concentration of 0.1 wt. % in the hydrogen peroxide solution, or zirconium dioxide nanoparticles at a concentration of 0.1% wt % in the hydrogen peroxide solution, or a 1:1 mixture of nanoporous cerium oxide nanoparticle macro-structures and zirconium dioxide nanoparticles, at a concentration of 0.1 wt. % in the hydrogen peroxide solution, were each observed to react with hydrogen peroxide and generate oxygen. With attention to FIG. 13B, it can be seen that the aforementioned nanoporous cerium oxide nanoparticle (NCeONP) macro-structures, zirconium dioxide nanoparticles and mixture of nanoporous cerium oxide nanoparticle (NCeONP) macro-structure with zirconium dioxide nanoparticles demonstrated an increase in cell viability of HEK293F cells when directly added to such cell culture medium. The concentration of the cerium oxide nanoparticles (NCeONP) macrostructures in a 1:1 mixture if zirconium dioxide nanoparticles was 300 micromolar (μM), 100 μM, 30 μM and 10 μM. Cell viability was then respectively observed to be 104%, 111%, 107% and 114%. The concentration of nanoporous cerium oxide nanoparticle (NCeONP) macro-structures was 300 μM, 100 μM, 30 μM and 10 μM. Cell viability was then respectively observed to be 118%, 112%, 108% and 106%. The concentration of zirconium dioxide nanoparticles was 300 μM, 100 μM, 30 μM and 10 μM. Cell viability was then respectively observed to be 113%, 130%, 127% and 123%.

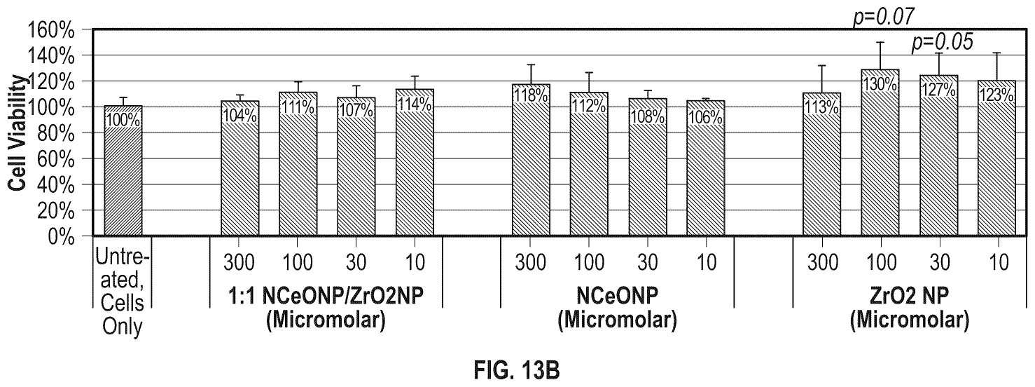

Example 5. With attention to FIG. 14, it can be seen that the aforementioned nanoporous cerium oxide nanoparticle (NCeONP) macro-structures, zirconium dioxide nanoparticles and mixture of nanoporous cerium oxide nanoparticle (NCeONP) macro-structure with zirconium dioxide nanoparticles demonstrated an increase in cell viability of HEK293F cells and CHO-S cell when directly added to such cell culture medium. The preferred concentration of the 1:1 mixture of cerium oxide nanoparticles (NCeONP) macrostructures and zirconium dioxide nanoparticles for promoting the highest cell growth of CHO-S cells was found to be 617 μM. Stimulated CHO-S cell growth at this concentration was 57% higher compared to the control. Under the same conditions the stimulated growth for HEK293 cells was found to be highest at a concentration of 1852 μM where a 21% increase in the cell viability was found.

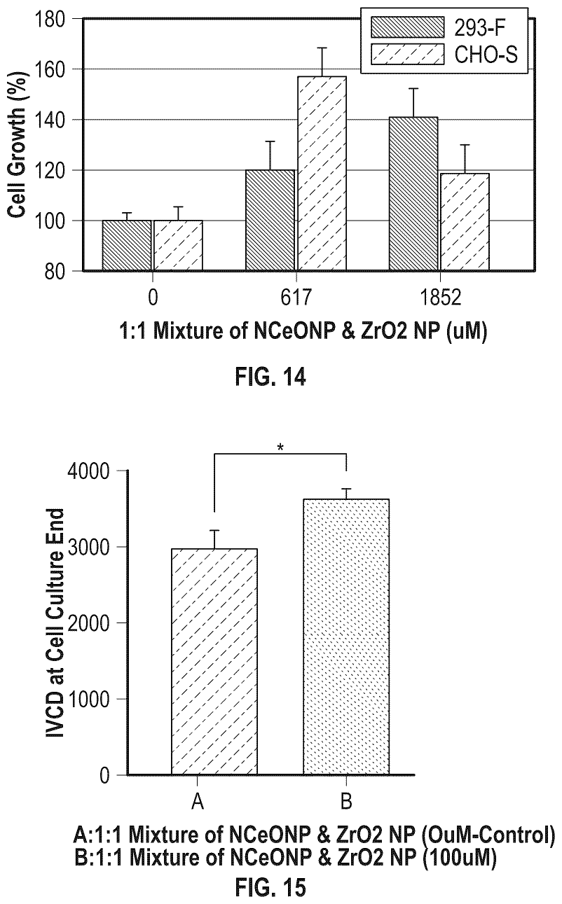

Example 6. While Example 5 demonstrates the growth of cells on Day 3 in a 96-well plate over three days, a biomanufacturing experiment was conducted in a 125 ml shake flask to determine IVCD (Integral Variable Cell Density). The Integral (area under the curve) of Viable Cell Density (IVCD) or concentration is an essential calculated metric in cell culture operations. IVCD quantifies the effective working time for a dynamic viable cell concentration within a given frame of time analogous to a calculation of man-hours. One vial of a CHO DG44 cell line was thawed into two 125-ml Optimum Growth® shake flasks (Thomson Instruments), containing 40 ml of eCHO medium (Lonza) supplemented with 8 mM Glutamax (Life Technologies) and 0.1% Anti-clumping agent (Life Technologies). Cells were routinely grown at 37° C. and 5% CO₂ on an orbital shaker at 125 rpm. Cells were passaged 2 times (approximately every 3 days) and then expanded into three 125-ml shake flasks. At inoculation, cells from the three flasks were combined and centrifuged and resuspended in complete eCHO medium as follows. The cell viability was 98%. 9×10\hat{ }6 cells were transferred to a 50 ml sterile conical tube and 8 identical tubes were prepared. Cells were centrifuged at 1200 rpm for 5 minutes, and the cell supernatant was discarded. 10 ml complete eCHO medium was added to each 50 ml tube, and cells were resuspended gently and then transferred to a 125-ml shake flask; an additional 35 ml complete eCHO medium was added for a final starting cell density of 2×10\hat{ }5/ml. The 1:1 mixture of NCeONP and ZrO₂ powders (non-sterile) were suspended in the medium to yield a final concentration of 100 μM. Cells were grown as described above. Samples were taken every other day for measurement of viability and viable cell density (using trypan blue staining and a TC10 automated cell counter), nutrient analysis and lactate dehydrogenase measurement. Cells were fed with 5% eCHO feed (2.2 ml) every other day, starting at day 3 after inoculation. Glucose, lactate, glutamine, and glutamate were measured using a YSI 2950 Biochemical analyzer. Lactate dehydrogenase was measured using LDH-Cytox Assay kit (Biolegend, Cat. 426401). The experiment was terminated on day 10 as the cell viability had dropped significantly. As seen from FIG. 15, an increase of about 20% in IVCD was observed in the presence of 1:1 mixture of NCeONP and ZrO₂ NP at 100 μM concentration.

Example 7. This is an example to demonstrate the efficacy of a 1:1 mixture of NCeONP and ZrO₂ NP to increase the production of monoclonal antibodies (mAB) in a 250 ml AMBR250 reactor system using a generic protocol described below.

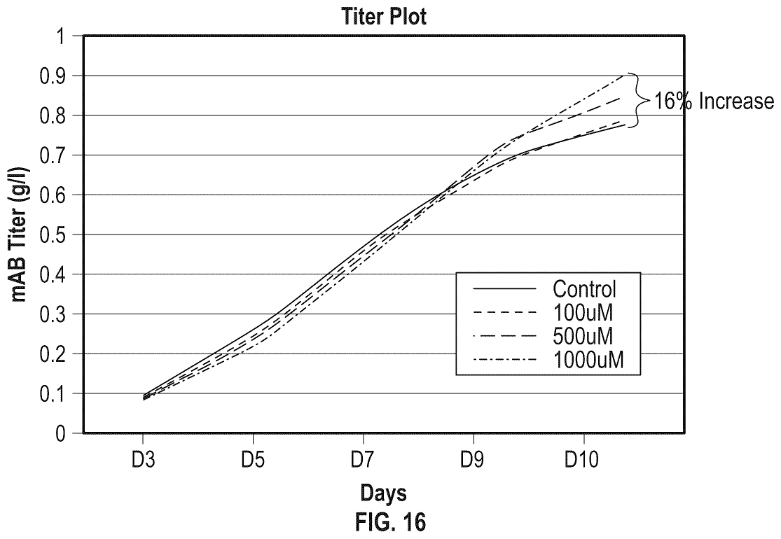

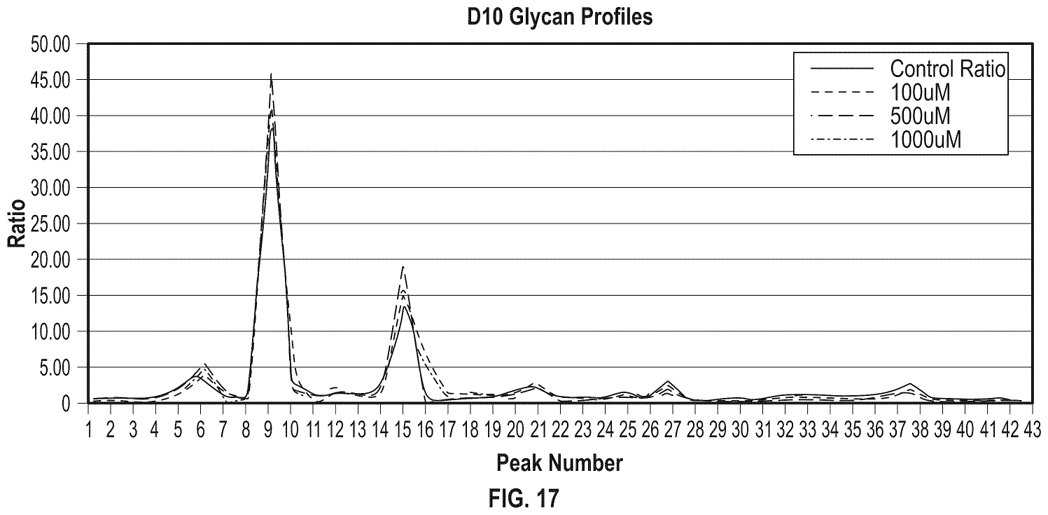

NIST CHO cell line, a clonal CHO-K1 cell line producing eNIST mAb, was prepared using commercially available EX-CELL® CD CHO Fusion media from MilliporeSigma (14365c). The AMBR250 reactor system was set up using the procedures and setting parameters described in the AMBR250 operation manual. The Dissolved Oxygen (DO) and pH are pre-calibrated within the vessels. The appropriate glucose, antifoam, and feed media, and base solutions were filled in the reservoirs attached to the vessel. The media and powder in different concentrations were added to different vessels in the biosafety cabinet while ensuring no contamination in any sources, including media and powder. The vessels were run for two days in AMBR250 system before inoculation and pH, DO, and any signs of contamination was carefully monitored. The vessels were then seeded with a single cell suspension (>10×106 cells/mL) at a final concentration of 0.5×106 cells/mL. The run continued until Day 10 for each condition. The standard harvesting and purification steps were carried out to measure the production of mAb as determined by the titer values. For example, The NOVA BioFlex was used for nutrient monitoring including cell count, viable cell density (VCD), cell viability (CV), glucose, lactate, glutamate, ammonium, and other ions on different days up to ten days of the experiment. The Octet BLI was used to measure mAb titer on each day. The HPLC was used to measure glycosylation, that shows the purity of mAB produced, at harvest. FIG. 16 shows increased mAb production with an increase in the concentration of the 1:1 mixture of NCeONP and ZrO₂ NP used. FIG. 17 shows that the glycosylation patterns or relative abundance of each glycan species remain the same, indicating that the quality of the increased production of mAb produced in the presence of a 1:1 mixture of NCeONP and ZrO₂ NP is similar to the one produced in control. Glycosylation is recognized as a critical quality attribute (CQA) for specific therapeutic antibodies, including both novel mAbs and biologics.

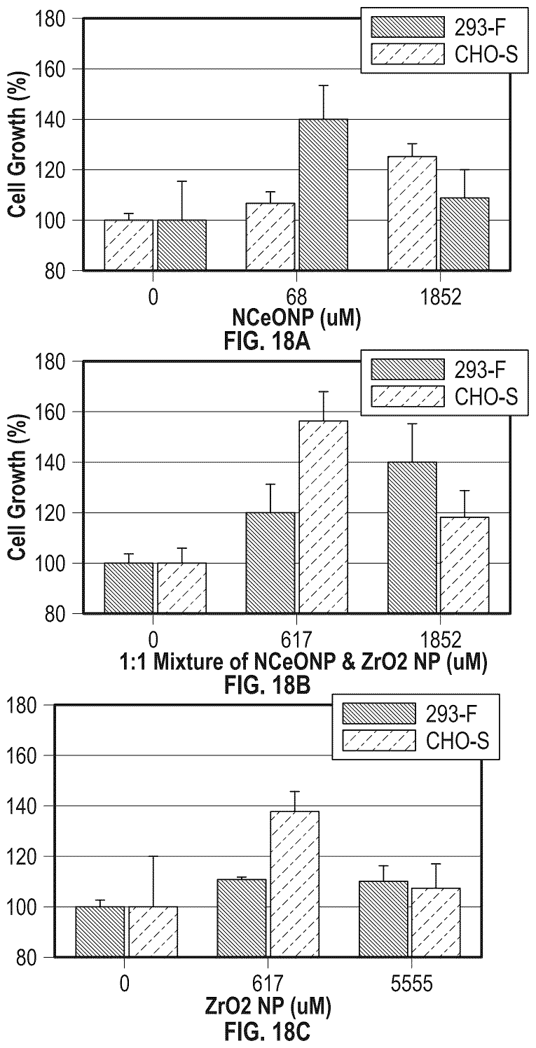

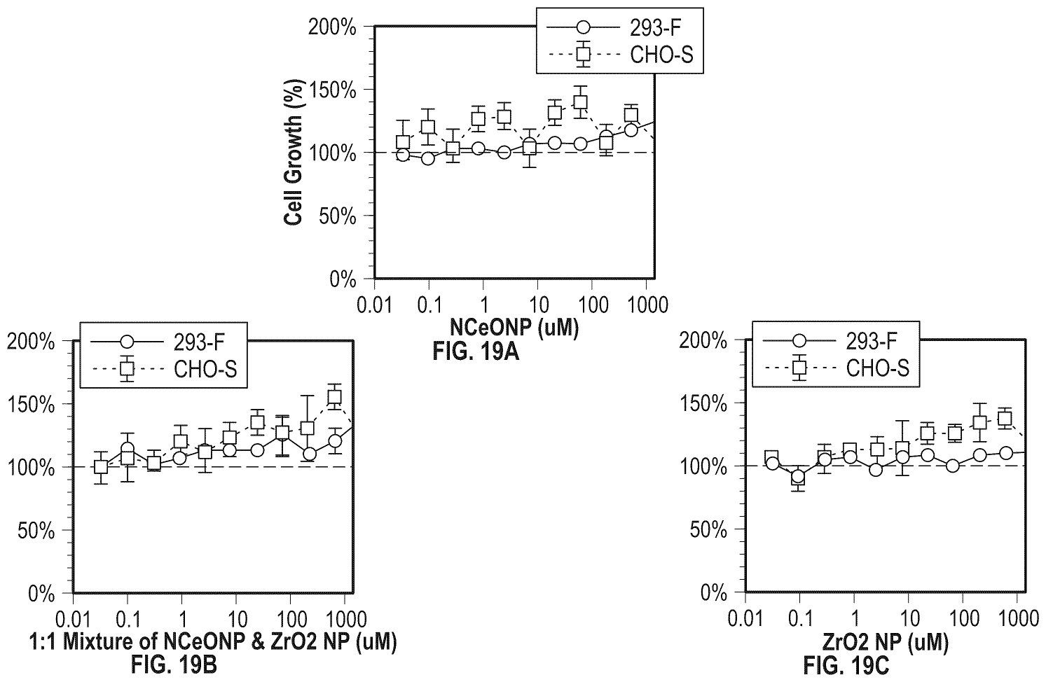

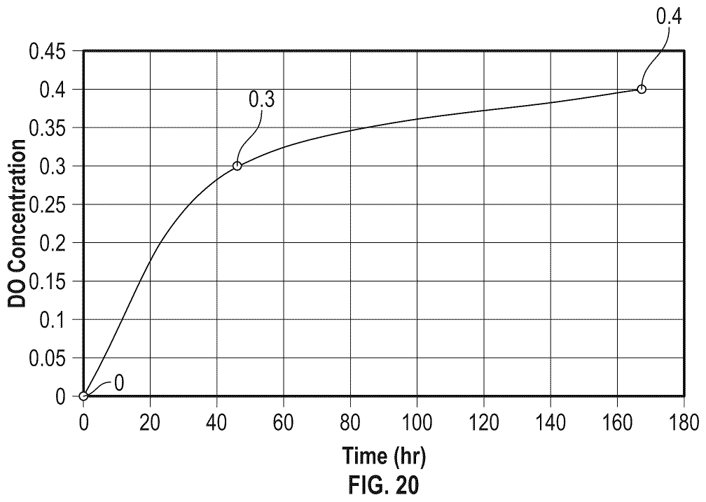

Example 8. A comparative analysis of the effect of NCeONP, ZrO₂ NP and a 1:1 mixture of NCeONP & ZrO₂ on the growth of HEK-293 and CHO-S cells using the protocol similar as described in Example 5, demonstrates a synergistic and enhanced ability of the mixture to stimulate the cell growth more than either of the individual components. As can be seen from the FIGS. 18A, 18B and 18C and FIGS. 19A, 19B and 19C, a dose-dependent comparative analysis of the impact of NCeONP and ZrO₂ NP shows that their combined effect (57% higher compared to control) is superior to each separately (40% for NCeONP and 38% for ZrO₂ NP higher compared to the control).

Examples 9. A bioprocessing bag was made using the films that generate dissolved oxygen (DO) as shown in FIGS. 12A and 12B. Such bags were used to grow cells for biomanufacturing. Bags were produced from such films obtained by compounding polymers such as EVA, PE, PP or a combination thereof or PVC with the exemplary and preferred 1:1 additive mixture of NCeONP and ZrO₂ NP. In one example, CHO cells were grown in a control bag made with the films without any additives and a test bag was made with films compounded with the preferred 1:1 mixture of NCeONP and ZrO₂ NP. The cell viability of CHO cells was also assessed after just five days of cell culturing and indicated the following results:

| Cultured CHO | | Test Bag (made with |
|---|---|---|
| Cells in the | Control Bag | film containing 2.5% |
| bioprocessing bag | (Film made without | of 1:1 mixture of |
| after five days | NCeONP/ZrO₂) | NCeONP/ZrO₂ NP) |
| Total number of Cells | 2.50 × 10⁶ Cells | 2.48 × 10⁶ Cells |
| Total number of Live | 2.40 × 10⁶ Cells | 2.43 × 10₆ Cells |
| Cells | | |
| Total percentage of | 96% | 98% |
| Viable Cells | | |

Example 9 demonstrates that the bioprocessing bag made from the films containing a 1:1 ratio of the additive NCeONP/ZrO₂ improved cell viability by 2% after just five days of culturing.

Example 10 demonstrates the nanoporous cerium oxide nanoparticle macro-structures and zirconium dioxide nanoparticles at a 1:1 ratio may be provided in a polymeric bead (ethylene 1-octene copolymer) at a 10.0% (wt.) loading where the polymeric bead is comprised of a porous shell to contain the additives. As illustrated, in the presence of hydrogen peroxide, the dissolved oxygen level increases over time.

What is claimed is:

1. A method of producing at least one biologically-produced product comprising:
providing a cell culture medium and at least a first type of biological cell configured to express at least one biologically-produced product;
placing said cell culture medium and said first type of biological cell in a bioreactor wherein the bioreactor provides at least one of cerium oxide nanoparticles, nanoporous cerium oxide nanoparticle macro-structures, or zirconium dioxide nanoparticles;
causing said first type of biological cell to express said at least one biological product.

2. The method of claim 1 wherein said nanoporous cerium oxide nanoparticle macro-structures have a pore size in the range of 10 nm to 1100 nm.

3. The method of claim 1 wherein said nanoporous cerium oxide nanoparticle macro-structures have a diameter in the range of 50 nm to 30,000 nm.

4. The method of claim 1 wherein said cerium oxide nanoparticles have a diameter in the range of 10 nm to 100 nm.

5. The method of claim 1 wherein said zirconium dioxide nanoparticles have a diameter in the range of 10.0 nm to 2000.0 nm.

6. The method of claim 1 wherein said at least one of cerium oxide nanoparticles, nanoporous cerium oxide nanoparticle macro-structures, or zirconium dioxide nanoparticles are present in said cell culture medium at a concentration of 1.0 nanomolar to 1000 millimolar.

7. The method of claim 1 wherein said bioreactor provides cerium oxide nanoparticles and nanoporous cerium oxide nanoparticle macro-structures.

8. The method of claim 1 wherein said bioreactor provides cerium oxide nanoparticles and zirconium dioxide nanoparticles.

9. The method of claim 1 wherein said bioreactor provides nanoporous cerium oxide nanoparticle macro-structures and zirconium dioxide nanoparticles.

10. The method of claim 9 wherein said bioreactor provides a 1:10 to 10:1 mixture of nanoporous cerium oxide nanoparticle macrostructures and zirconium dioxide nanoparticles.

11. The method of claim 10 wherein said bioreactor provides a 1:1 mixture of nanoporous cerium oxide nanoparticle macro-structures and zirconium dioxide nanoparticles.

12. The method of claim 1 wherein said cell culture medium produces a reactive oxygen species and said at least one of said cerium oxide nanoparticles, nanoporous cerium oxide nanoparticle macro-structures, or zirconium dioxide nanoparticles produce oxygen from said reactive oxygen species.

13. The method of claim 1 wherein said cell culture medium contains free radicals and at least one of said cerium oxide nanoparticles, nanoporous cerium oxide nanoparticle macro-structures, or zirconium dioxide nanoparticles scavenge said free radicals.

14. The method of claim 1 wherein said bioreactor has an inner surface in contact with said cell culture medium and at least one of cerium oxide nanoparticles, nanoporous cerium oxide nanoparticle macro-structures, or zirconium dioxide nanoparticles is present as a coating on said bioreactor inner surface.

15. The method of claim 1 wherein at least one of cerium oxide nanoparticles, nanoporous cerium oxide nanoparticle macro-structures, or zirconium dioxide nanoparticles are loaded into a polymeric film wherein said loaded polymeric film is positioned within said bioreactor.

16. A method of producing at least one biologically-produced product comprising:
providing a cell culture medium and at least a first type of biological cell configured to express at least one biologically-produced product;
placing said cell culture medium and said first type of biological cell in a bioreactor comprising polymer film containing at least one of cerium oxide nanoparticles, nanoporous cerium oxide nanoparticle macro-structures, or zirconium dioxide nanoparticles;
causing said first type of biological cells to express said at least one biological product.

17. The method of claim 16 wherein said nanoporous cerium oxide nanoparticle macro-structures have a pore diameter in the range of 10 nm to 1100 nm.

18. The method of claim 16 wherein said nanoporous cerium oxide nanoparticle macro-structures have a diameter in the range of 50 nm to 30,000 nm.

19. The method of claim 16 wherein said polymer film has a thickness in the range of 0.01 mm to 10.0 mm.

20. The method of claim 16 wherein said polymer film is selected from the group consisting of poly(vinyl chloride), polyethylene, polypropylene, ethylene-propylene copolymers, polyethylene-co-vinyl acetate, polyester, polyamide, cellulose, or polyurethane.

21. The method of claim 16 wherein said at least one of said cerium oxide nanoparticles or cerium oxide nanoparticle macro-structures or zirconium dioxide nanoparticles are present in said polymer film at a level of 0.01 wt. % to 10.0 wt. %.

22. The method of claim 16 wherein two or more of said cerium oxide nanoparticles, cerium oxide nanoparticle macro-structures or zirconium dioxide nanoparticles are mixed and present at a combined level in said polymer film at a level of 0.01 wt. % to 10.0 wt. %.

23. The method of claim 16 wherein said cell culture medium produces a reactive oxygen species and said polymer film containing at least one of cerium oxide nanoparticles, nanoporous cerium oxide nanoparticle macro-structures or zirconium dioxide nanoparticles, produces oxygen from said reactive oxygen species.

24. The method of claim 16 wherein said cell culture medium contains free radicals polymer and said polymer film containing at least one of cerium oxide nanoparticles, nanoporous cerium oxide nanoparticle macro-structures, or zirconium dioxide nanoparticles scavenge said free radicals.

25. The method of claim 16 wherein said polymer film containing at least one of cerium oxide nanoparticles, nanoporous cerium oxide nanoparticle macro-structures, or zirconium dioxide nanoparticles comprises a mixture of cerium oxide nanoparticles and zirconium dioxide nanoparticles.

26. The method of claim 16 wherein said polymer film containing at least one of cerium oxide nanoparticles, nanoporous cerium oxide nanoparticle macro-structures, or zirconium dioxide nanoparticles comprises a mixture of nanoporous cerium oxide nanoparticle macro-structures and zirconium dioxide nanoparticles.

27. The method of claim 26 wherein said mixture of nanoporous cerium oxide nanoparticle macro-structure and zirconium oxide nanoparticles comprises a 1:10 to 10:1 mixture of nanoporous cerium oxide nanoparticle macrostructures and zirconium dioxide nanoparticles.

28. The method of claim 27 wherein said mixture of nanoporous cerium oxide nanoparticle macro-structure and zirconium oxide nanoparticles comprises a 1:1 mixture of nanoporous cerium oxide nanoparticle macrostructures and zirconium dioxide nanoparticles.

29. A method for growth/expansion of at least one biological cell comprising:
providing a cell culture medium and at least a first type of biological cell for growth/expansion;
placing said cell culture medium and said first type of biological cell in a bioreactor wherein the bioreactor provides at least one of cerium oxide nanoparticles, nanoporous cerium oxide nanoparticle macro-structures, or zirconium dioxide nanoparticles;
causing said first type of biological cell to undergo growth/expansion.

30. The method of claim 29 wherein said nanoporous cerium oxide nanoparticle macro-structures have a pore size in the range of 10 nm to 1100 nm.

31. The method of claim 29 wherein said nanoporous cerium oxide nanoparticle macro-structures have a diameter in the range of 50 nm to 30,000 nm.

32. The method of claim 29 wherein said cerium oxide nanoparticles have a diameter in the range of 10 nm to 100 nm.

33. The method of claim 29 wherein said zirconium dioxide nanoparticles have a diameter in the range of 10.0 nm to 2000.0 nm.

34. The method of claim 29 wherein said at least one of cerium oxide nanoparticles, nanoporous cerium oxide nanoparticle macro-structures, or zirconium dioxide nanoparticles are present in said cell culture medium at a concentration of 1.0 nanomolar to 1000 millimolar.

35. The method of claim 29 wherein said bioreactor provides cerium oxide nanoparticles and nanoporous cerium oxide nanoparticle macro-structures.

36. The method of claim 29 wherein said bioreactor provides cerium oxide nanoparticles and zirconium dioxide nanoparticles.

37. The method of claim 29 wherein said bioreactor provides nanoporous cerium oxide nanoparticle macro-structures and zirconium dioxide nanoparticles.

38. The method of claim 37 wherein said bioreactor provides a 1:10 to 10:1 mixture of nanoporous cerium oxide nanoparticle macrostructures and zirconium dioxide nanoparticles.

39. The method of claim 38 wherein said bioreactor provides a 1:1 mixture of nanoporous cerium oxide nanoparticle macro-structures and zirconium dioxide nanoparticles.

40. The method of claim 29 wherein said cell culture medium produces a reactive oxygen species and said at least one of said cerium oxide nanoparticles, nanoporous cerium oxide nanoparticle macro-structures, or zirconium dioxide nanoparticles produce oxygen from said reactive oxygen species.

41. The method of claim 29 wherein said cell culture medium contains free radicals and at least one of said cerium oxide nanoparticles, nanoporous cerium oxide nanoparticle macro-structures, or zirconium dioxide nanoparticles scavenge said free radicals.

42. The method of claim 29 wherein said bioreactor has an inner surface in contact with said cell culture medium and at least one of cerium oxide nanoparticles, nanoporous cerium oxide nanoparticle macro-structures, or zirconium dioxide nanoparticles is present as a coating on said bioreactor inner surface.

43. The method of claim 29 wherein at least one of cerium oxide nanoparticles, nanoporous cerium oxide nanoparticle macro-structures, or zirconium dioxide nanoparticles are loaded into a polymeric film wherein said loaded polymeric film is positioned within said bioreactor.

44. A biomanufacturing system comprising:
a bioreactor providing at least one of cerium oxide nanoparticles, nanoporous cerium oxide nanoparticle macro-structures, or zirconium dioxide nanoparticles; and
a cell culture medium and at least a first type of biological cell contained in said bioreactor configured to express at least one biologically-produced product.

45. The biomanufacturing system of claim 29 wherein said at least one of cerium oxide nanoparticles, nanoporous cerium oxide nanoparticle macro-structures, or zirconium dioxide nanoparticles are present in said cell culture medium at a concentration of 1.0 nanomolar to 1000 millimolar.

46. The biomanufacturing system of claim 29 wherein said at least one of cerium oxide nanoparticles, nanoporous cerium oxide nanoparticle macro-structures or zirconium dioxide nanoparticles are present as a coating on an internal surface of said bioreactor.

47. The biomanufacturing system of claim 29 wherein the cerium oxide nanoparticles and/or nanoporous cerium oxide nanoparticle macro-structures and/or zirconium dioxide nanoparticles are configured to convert reactive oxygen species within said cell culture medium to oxygen and/or scavenge free radicals.

48. A biomanufacturing system comprising:
a bioreactor containing a cell culture medium and at least a first type of biological cell configured to express at least one biologically-produced product; and
said bioreactor including a polymer film containing at least one of cerium oxide nanoparticles and/or nanoporous cerium oxide nanoparticle macro-structures and/or zirconium dioxide nanoparticles.

49. The biomanufacturing system of claim 33 wherein said polymer film containing cerium oxide nanoparticles and/or nanoporous cerium oxide nanoparticle macro-structures and/or zirconium dioxide nanoparticles is configured to contact said cell culture medium and convert reactive oxygen species within said cell culture medium to oxygen and/or scavenge free radicals.

50. A bioreactor comprising polymer film containing at least one of cerium oxide nanoparticles, nanoporous cerium oxide nanoparticle macro-structures or zirconium dioxide nanoparticles.

51. The bioreactor of claim 35 wherein said polymer film contains a mixture of cerium oxide nanoparticles and zirconium dioxide nanoparticles.

52. The bioreactor of claim 35 wherein said polymer film contains a mixture of nanoporous cerium oxide nanoparticle macro-structures and zirconium dioxide nanoparticles.

53. The bioreactor of claim 37 wherein said polymer film provides a 1:10 to 10:1 mixture of nanoporous cerium oxide nanoparticle macrostructures and zirconium dioxide nanoparticles.

54. The bioreactor of claim 38 wherein said polymer film provides a 1:1 mixture of nanoporous cerium oxide nanoparticle macro-structures and zirconium dioxide nanoparticles.

55. A bioreactor comprising polymer film containing at least one of cerium oxide nanoparticles, nanoporous cerium oxide nanoparticle macro-structures or zirconium dioxide nanoparticles.
